(19) United States
(12) Patent Application Publication
(10) Pub. No.: US 2026/0047959 A1
(43) Pub. Date: Feb. 19, 2026

(54) SAFETY GLASS SYSTEM

(71) Applicant: Brandon Bradley Gordon-Lalonde, Kingston (CA)

(72) Inventor: Brandon Bradley Gordon-Lalonde, Kingston (CA)

(21) Appl. No.: 18/807,359

(22) Filed: Aug. 16, 2024

(51) Int. Cl. A61F 9/02; G06F 16/41; G06V 20/40; H04N 5/76

(52) U.S. Cl. CPC A61F 9/029; G06F 16/41; G06V 20/40; H04N 5/76; G06V 2201/08

(57) ABSTRACT

A safety glass system that incorporates a unique pair of safety glasses and a method of utilization wherein the safety glasses are communicably coupled to a software database that facilitates the method of the present invention. The system includes a pair of safety glasses having element such as but not limited to video cameras, light members, audio members and a transceiver. The method of the system includes the step of recording and capturing an audio-visual datafile wherein the captured datafile is transmitted to the software database. The software database is employed for a plurality of alternate tasks to include parts identification and further include specification analysis on an object in a frame from the captured audio-visual datafile. The software database transmits information pertaining to the object in the capture audio-visual datafile wherein at least some of the information can be broadcast to a user via the audio members.

### FIELD OF THE INVENTION

The present invention relates generally to safety equipment, more specifically but not by way of limitation, a safety glass system that includes safety glasses configured to record an audio-visual data file during events such as but not limited to facilitating a vehicle repair. The safety glasses of the present invention are further configured to provide visual inspection of parts tolerance and compare against a database for identification of an out of specification element.

### BACKGROUND

Documentation of repairs has become expected by many customers of various vendors such as but not limited to vehicle repair shops. In some areas, video documentation is required by law and the vendor must provide video documentation of the broken part and/or issue with the vehicle or item being repaired. In many environments, the individuals facilitating a repair or inspecting the vehicle or other item for damage has their hands occupied with tools or other equipment and is unable to manage an additional device that would provide video recording of the repair and/or inspection.

Many other environments such as but not limited to factories, warehouses and other types of facilities require the wearing of safety equipment such as but not limited to safety glasses. In these types of commercial environments there are strict compliance regulations for workplace safety. While some environments are monitored with conventional cameras to document working conditions as well as worker safety, there are many environments that are not documented with video data files. Another deficiency with current equipment is the inability to control documentation that is specifically associated with an identifier such as but not limited to a vehicle identification number.

Accordingly, there is a need for a safety glass system that is configured to record an audio-visual data file that can be employed for such purposes as but not limited to: repair documentation, inspection, parts identification and out of tolerance review.

### SUMMARY OF THE INVENTION

It is the object of the present invention to provide a safety glass system and method that is operable to provide an audio-visual data file to be employed for multiple purposes wherein the present invention includes safety glasses having at least one camera and a microphone.

Another object of the present invention is to provide a method configured to facilitate video documentation of a repair or inspection wherein the present invention includes a database communicably coupled to the safety glasses.

A further object of the present invention is to provide a safety glass system and method that is operable to provide an audio-visual data file to be employed for multiple purposes wherein the safety glasses further include audio earphones operably coupled to the safety glasses.

Yet a further object of the present invention is to provide a method configured to facilitate video documentation of a repair or inspection wherein the method of the present invention includes a step of comparing a part being viewed by a wearer of the safety glasses with a data file in the database for identification thereof.

Still another object of the present invention is to provide a safety glass system and method that is operable to provide an audio-visual data file to be employed for multiple purposes wherein the at least one camera mounted to the safety glasses could be movably mounted.

An additional object of the present invention is to provide a method configured to facilitate video documentation of a repair or inspection wherein the method of the present invention includes a step of uploading the captured audio-visual data file to the database.

Yet a further object of the present invention is to provide a safety glass system and method that is operable to provide an audio-visual data file to be employed for multiple purposes wherein the safety glasses include a visual communication indicator light.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is a right front perspective of an embodiment of safety glasses of the present invention; and

FIG. 2 is a left front perspective of an embodiment of safety glasses of the present invention; and

FIG. 3 is a flowchart of an exemplary operational method of the present invention.

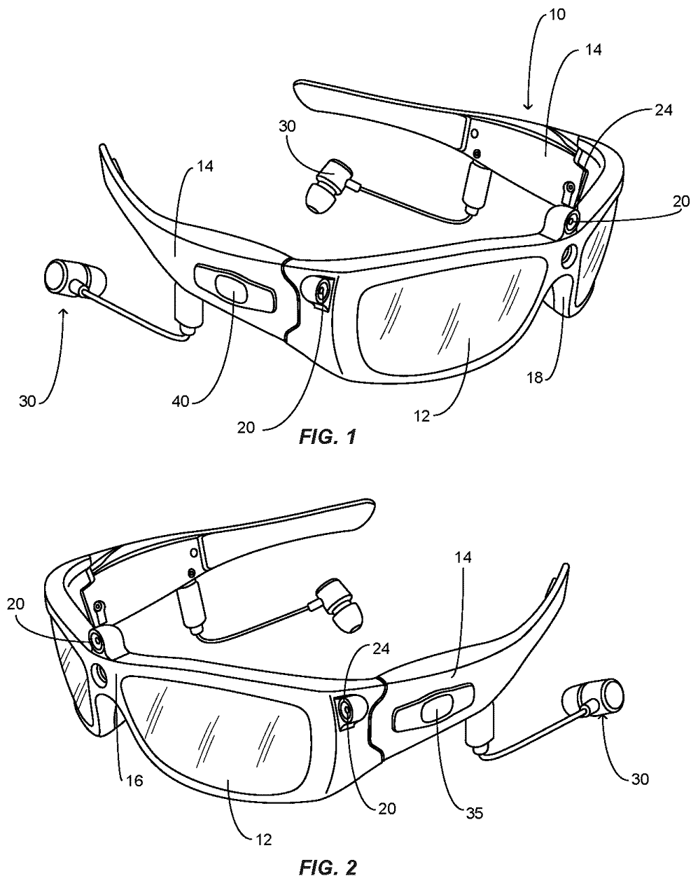

### DETAILED DESCRIPTION

Referring now to the drawings submitted herewith, wherein various elements depicted therein are not necessarily drawn to scale and wherein through the views and figures like elements are referenced with identical reference numerals, there is illustrated a safety glass system 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

Referring in particular to the Figures submitted herewith, the safety glass system 100 includes at least one pair of safety glasses 10. The safety glasses 10 are constructed of a high impact durable material and include the traditional construction elements of conventional glasses such as lenses 12, temple members 14, bridge 16 and nose pad members 18. It should be understood within the scope of the present invention that the embodiment of the safety glasses 10 illustrated herein is exemplary only and that it is contemplated within the scope of the present invention that the safety glasses 10 could be constructed in alternate styles within the scope of the present invention.

The safety glasses 10 includes cameras 20 wherein the cameras 20 are conventional video cameras that are equipped with the necessary electronics to capture video images along with audio. In a preferred embodiment of the present invention, the safety glasses 10 include cameras located proximate the bridge 16 and proximate each lens 12 in order to capture what the wearer of the safety glasses 10 is looking at or the area in which the wearer is looking. While three cameras 20 are illustrated herein, it is contemplated within the scope of the present invention that the safety glasses 10 could have more than three or less than three cameras 20 in order to capture the desired visual data files. The safety glasses 10 includes light members 24 that are encased in the camera housing being circumferentially disposed around the camera 20. Light members 24 are conventional LED lights and are operable to emit light for effective video capture. It is contemplated within the scope of the present invention that the safety glasses 10 could employ various quantities of light members 24 and further that the light members 24 could be mounted in alternate locations.

The safety glasses 10 includes audio members 30. Audio members 30 are operably coupled to the temple members 14 of the safety glasses 10 and are configured to emit audio data into the ears of the wearer of the safety glasses 10. The audio members 30 broadcast audio from the video data file being captured as well as audio data from a database(not illustrated herein) with which the safety glasses 10 are communicably coupled as is further discussed herein. The safety glasses 10 includes a power supply 35 configured to provide the necessary power for operation of the safety glasses. A transceiver 40 is mounted within the temple member 14 of the safety glasses 10. The transceiver 40 includes the necessary electronics to transmit, receive, store and manipulate video and audio data. The transceiver 40 facilitates the communicable coupling with the database of the safety glass system 100.

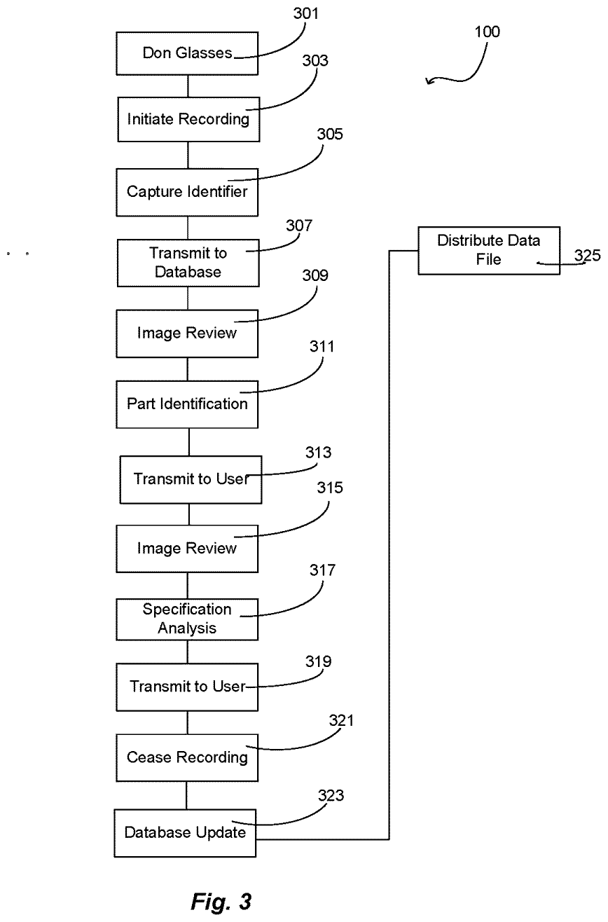

Referring now to FIG. 3, an exemplary operational method of the safety glass system 100 is diagrammed therein. It should be understood that the method outlined in FIG. 3 is exemplary and that the steps thereof could be execute in an alternate order or have some thereof be eliminated during use of the safety glass system 100. In step 301, a user will don the safety glasses 10. Step 303, the user will initiate a video recording utilizing the cameras 20 wherein any associated audio is captured to comprise an audio-visual datafile. In step 305, the user will capture an image of a unique identifier that is employed to associate the captured audio-visual data file with a particular object such as but not limited to a vehicle. By way of example but not limitation, the unique identifier could be a QR code or a vehicle identification number. This unique identifier provides a technique to catalog the stored audio-visual datafile.

In step 307, the audio-visual datafile is continuously generated and transmitted to the database of the present invention. The database stores the captured audio-visual datafile as the datafile is captured. In step 309, at least one frame of the captured audio-visual datafile is reviewed. Review of the frame of the audio-visual datafile occurs upon request of the user and is executed utilizing suitable automated protocols. In an exemplary analysis review of the frame of the captured audio-visual datafile, the frame contains a graphical image of a part that the user desires to obtain additional information thereon. In step 311, the database provides identification of the part in the image of the frame of the captured audio-visual datafile. The identification can include but is not limited to information such as the part number, cost and availability thereof. In step 313, the database transmits to the user the information about the part wherein the audio members 30 can be employed to receive requested data about the part in the image. In step 315, the database reviews an alternate image captured from the audio-visual datafile generated by the safety glasses 10. Step 317, the database executes either an analysis, utilizing suitable software tools, or a comparison of a like image wherein the objective of the aforementioned is to perform a specification analysis either on the entire object or a portion thereof. By way of example but not limitation, a portion of the image could be measured to ensure a proper tolerance for a part or to determine if wear on the part has occurred and needs to be replaced. It is contemplated within the scope of the present invention that the software in the database could have cataloged therein multitudes of images and/or specifications so as to perform the desired task on the image from the audio-visual datafile.

In step 319, the software database will transmit to the user of the safety glasses 10 information regarding the specification analysis wherein this information can be received and broadcast to the user via the audio members 30. In step 321, a user will elect to cease recording and as such capture of the audio-visual datafile is halted. Step 323, the complete audio-visual datafile recorded by the safety glasses 10 is uploaded to the database and has a filename or tag associated therewith that includes the identifier from step 305 so as to maintain traceable records thereof. In step 325, the datafile can be distributed utilizing suitable electronic techniques. While one pair of safety glasses 10 has been discussed herein, it is contemplated within the scope of the present invention that more than one pair of safety glasses 10 could be executing the method of the present invention simultaneously.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

1. A method of employing safety glasses configured to record an audio-visual datafile wherein the safety glass method comprises the steps of:
providing at least one pair of safety glasses, said at least one pair of safety glasses having temple members and lenses, said at least one pair of safety glasses having at least one video camera, said at least one video camera configured to capture the audio-visual datafile, said at least one pair of safety glasses having a transceiver, said transceiver having electronics to receive, store, transmit and manipulate data, said at least one pair of safety glasses having a power supply,
donning the at least one pair of safety glasses, wherein a user will don the at least one pair of safety glasses,
initiating a recording, wherein the recording commences capture of the audio-visual datafile, the audio-video datafile comprising an unaltered audio-visual feed captured from a viewpoint of the user;
transmitting at least a portion of the audio-visual datafile, wherein the at least one pair of safety glasses transmits the at least a portion of the audio-visual datafile to a software database;
reviewing the at least a portion of the audio-visual datafile, wherein the software database provides a review of the image in the at least a portion of the audio-visual datafile, the review comprising a comparison one or more objects in the image with one or more reference images;
transmitting information to the user, wherein the information pertains to the review of the image in the at least a portion of the audio-visual datafile;
ceasing the capture of the audio-visual datafile; and
storing the audio-visual datafile, wherein the audio-visual datafile is stored in the software database.

2. The method of employing safety glasses configured to record an audio-visual datafile as recited in claim 1, and further including a step of capturing a unique identifier, wherein the unique identifier is specific to an object being captured in the audio-visual datafile.

3. The method of employing safety glasses configured to record an audio-visual datafile as recited in claim 2, and further including in the step of performing the image review, reviewing the object captured in the audio-visual datafile for one or more of: type of the object, wear on the object, and fit tolerance of the object.

4. The method of employing safety glasses configured to record an audio-visual datafile as recited in claim 3, and further including a step of providing parts identification for the object in the audio-visual datafile.

5. The method of employing safety glasses configured to record an audio-visual datafile as recited in claim 4, and further including a step of transmitting parts identification information to the user of the at least one pair of safety glasses.

6. The method of employing safety glasses configured to record an audio-visual datafile as recited in claim 5, and further including a step of performing a specification analysis, wherein the specification analysis is performed on the object in the audio-visual datafile.

7. The method of employing safety glasses configured to record an audio-visual datafile as recited in claim 6, and further including a step of transmitting specification analysis results to the user of the at least one pair of safety glasses.

8. The method of employing safety glasses configured to record an audio-visual datafile as recited in claim 7, and further including a step of storing a completed audio-visual datafile, said completed audio-visual datafile being stored in the software database and having the unique identifier associated therewith.
